(19) United States
(12) Patent Application Publication
(10) Pub. No.: US 2025/0152648 A1
(43) Pub. Date: May 15, 2025

(54) SYSTEM AND METHOD OF PROCESSING HEMP FOR EXTRACTION OF SELECT COMPONENTS THEREOF

(71) Applicant: Daniel Webster, Saratoga Springs, UT (US)

(72) Inventor: Daniel Webster, Saratoga Springs, UT (US)

(21) Appl. No.: 18/946,565

(22) Filed: Nov. 13, 2024

Related U.S. Application Data
(60) Provisional application No. 63/598,872, filed on Nov. 14, 2023.

(51) Int. Cl. A61K 36/185; B01D 21/26; B30B 1/18

(52) U.S. Cl. CPC A61K 36/3482; B01D 21/262; B30B 1/18; A61K 2236/15; A61K 2236/39; A61K 2236/51; A61K 2236/53; A61K 2236/55

(57) ABSTRACT

A method of processing hemp to obtain a desired hemp component includes providing a liquid solution including hemp into a press. The press is used to separate the liquid solution into a liquid faction and a solid faction, with the solid faction including hemp. A centrifuge is used to separate the liquid faction into heavy materials and a clarified liquid. The clarified liquid is the separated into waste and a desired component.

### CROSS-REFERENCE TO RELATED APPLICATIONS

This application claims the benefit of U.S. Application Ser. No. 63/598,872, filed Nov. 14, 2023, the contents of which are expressly incorporated herein by reference.

### STATEMENT RE: FEDERALLY SPONSORED RESEARCH/DEVELOPMENT

Not Applicable

### BACKGROUND

### 1. Technical Field

The present disclosure relates to a hemp processing methodology, and more specifically, the a method of efficiently processing hemp to extract valuable components therefrom, such as lignin, protein, sugars, omega-3 fatty acids, chlorophyll, and terpenes.

### 2. Description of the Related Art

Hemp is a plant that has a long history of industrial and consumable use. Along these lines, it is believed that 50,000 years ago, hemp was spun into usable fiber. Today, hemp is commonly used for fiber, cannabinoids, and for various building and agricultural purposes. However, the traditional methods of processing hemp often result in the undesirable loss of valuable components that can have various industrial and nutritional applications.

Accordingly, there is a need in the art for an improved method of processing hemp to extract desired components therefrom. Various aspects of the present disclosure address this particular need, as will be discussed in more detail below.

### BRIEF SUMMARY

In accordance with one embodiment of the present disclosure, there is provided a method of processing hemp to obtain a desired hemp component. The method includes providing a liquid solution including hemp into a press. The press is used to separate the liquid solution into a liquid faction and a solid faction, with the solid faction including hemp. A centrifuge is used to separate the liquid faction into heavy materials and a clarified liquid. The clarified liquid is the separated into waste and a desired component.

The method may also include the step of creating the liquid solution by cutting the hemp to release natural liquids from the hemp. The liquid solution may be created independent of any additives other than the hemp. The liquid solution may include a liquid component comprised solely of natural liquids in the hemp.

The method may further include the step of chopping the hemp into pieces.

The hemp used in the liquid solution may include less than 1% cannabidiol (CBD).

The press may be a screw press.

The method may also include the step of directing air under pressure within the press to keep the press clear of obstruction.

The step of providing the liquid solution may include providing chopped hemp into the press by a conveyor.

The method may additionally comprise the step of setting the press to a specific pressure based on a moisture content of hemp loaded into the press. The method may further comprise the step of obtaining moisture content data of the hemp loaded into the press from a moisture sensor. The method may additionally comprise the step of autonomously setting the press to the specific pressure in response to the moisture content data received from the moisture sensor.

The present disclosure will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is a schematic overview of a hemp processing system according to one embodiment; and

FIG. 2 is a flow chart associated with a hemp processing methodology according to one embodiment.

Common reference numerals are used throughout the drawings and the detailed description to indicate the same elements.

### DETAILED DESCRIPTION

The detailed description set forth below in connection with the appended drawings is intended as a description of certain embodiments of a hemp processing system and related methodology and is not intended to represent the only forms that may be developed or utilized. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual such relationship or order between such entities.

Hemp is a valuable resource having several desirable qualities and components. Several prior art techniques have been developed for processing the hemp to extract desired components from hemp. In most cases, conventional processing techniques require that the hemp is first dried before extractions can take place. Attempts have been made for wet processing of the hemp, although such techniques have not yielded positive results. In most conventional techniques, substances or additives were added to the water, in which wet processing of hemp, was attempted. However, the inclusion of such additives did not produce positive results. In fact, the addition of the additives added expense and complication to the hemp processing, without yielding any appreciable benefits.

Accordingly, various aspects of the present disclosure are directed toward processing wet hemp without the addition of additives. In this regard, it is understood that the hemp itself may include natural oils and/or other inherent components that, under certain processing conditions, may facilitate the wet processing of the hemp, and thus, may render additives unnecessary.

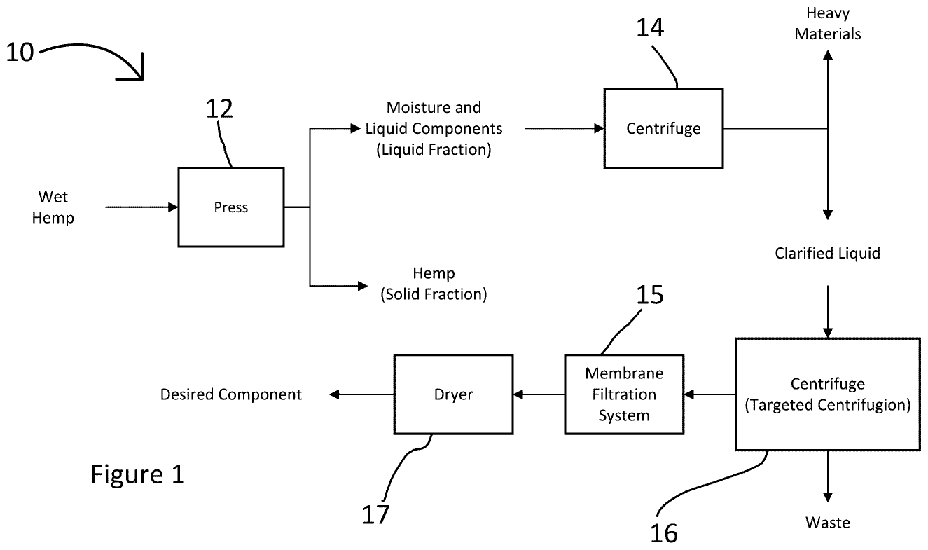

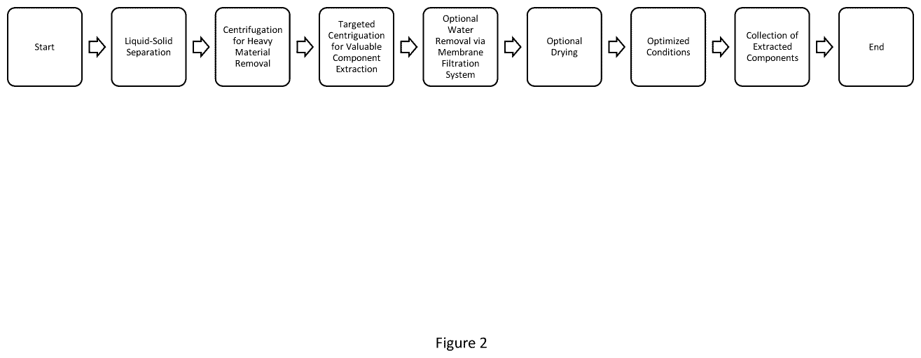

Referring now the drawings, wherein the showings are for purposes of illustrating preferred aspects of the present disclosure, and are not for purposes of limiting the same, FIG. 1 shows a system level overview of an exemplary hemp processing system 10, while FIG. 2 is a flow chart of an exemplary methodology associated with processing the hemp.

The hemp used in the process described herein may include industrial hemp of less than 1% cannabidiol (CBD) with non-detectable levels of tetrahydrocannabinol (THC). The hemp may be harvested between 45-60 days after planting, or alternatively, at full vegetative phase and in the initial stages of flower formation, but before reproductive maturity.

A wet hemp solution is created by chopping or cutting hemp leaves or other portions of a hemp plant into pieces. In one embodiment, the pieces may chopped with a silage or mint chopper and cut into pieces that are approximately 0.5-2.0 inch pieces. In this regard, each piece may have a length and width between 0.5-2.0 inches. In other words, no dimension of the piece may be greater than 2.0 inches, or less than 0.5 inches (other than the thickness). When a typical hemp plant is harvested, the moisture content is typically around 60%-80%; however, it is understood that the scope of the present disclosure is not limited to that particular moisture content. As the hemp is cut or chopped, a portion of the liquid from the hemp plant is released to form the wet hemp solution.

The wet hemp solution is then loaded into a press 12 to separate a liquid fraction of the solution from a solid fraction of the solution. In one embodiment, the chopped hemp may be continuously fed into the press 12 by a conveyor. The liquid fraction may include components of interest that are water-soluble and suspended in the liquid, while the solid fraction includes wet hemp. In one particular embodiment, the press 12 is a screw press having a large screw coupled to a press plate. Air nozzles may be used inside of the press 12 to keep the press 12 clear of obstruction to enable continuous flow. As the screw is rotated, the press plate may compress the wet hemp solution to output both a liquid portion and a solid portion. In particular, the press 12 may urge the liquid portion through a screen, while the solid portion remains on another side of the screen (e.g., the solid portion does not pass through the screen). In this regard, the screen may be suitably sized and configured to allow for such solid-liquid separation. The press 12 may be set to a specific pressure (e.g. PSI) and frequency (e.g., hertz setting) based on the moisture content of the raw biomass and based on the intended end use of the product. A moisture sensor may be used to measure the moisture content of the raw biomass and provide feedback data including the sensed moisture to a press controller, which may adjust the PSI and hertz setting based on the sensed moisture, as well as preprogrammed instructions. The moisture sensor may include a processor, memory (RAM or ROM) and other hardware necessary for implementing the functionalities described herein.

The solid fibrous material obtained as the solid portion output from the press 12 may be dried and chopped into homogenized pieces (e.g., pieces that are roughly the same size, that is, having a surface area that is within 10% of each other) that are used as filled in an end product and/or as a binder.

The liquid fraction obtained from the liquid-solid separation is further processed to initially homogenize the liquid fraction through cyclonic agitation. The liquid fraction may then be pumped into a centrifuge 14 for heavy material removal. The liquid fraction that is pumped into the centrifuge 14 may be approximately 0.8% suspended solids that are below 2 microns in size and approximately 5.2% dissolved solids. Any portion of this process may be repeated if necessary to achieve these specifications in the liquid faction.

The centrifuge 14 may be a solid bowl centrifuge, or other centrifuges known by those skilled in the art. The rate at which the liquid fraction is pumped into the centrifuge 14 may be approximately 10 gallons per minute (e.g., between 9-11 gallons per minute). The centrifuge 14 is designed to effectively remove heavy materials such as larger particulates, debris, and undesirable substances that may be present in the liquid fraction. According to one embodiment, the centrifuge 14 may be operated at a speed which generates a force having a magnitude of approximately 600-1400 G-forces, and more particularly 1000 G-forces. According to another embodiment, the centrifuge 14 may be set to operate at approximately 3250 rpm, or 2100 Gs.

The centrifuge 14 may produce two general outputs, namely, a solid output (e.g., hemp particulates, sand, dirt, etc.), which may be ejected out of the centrifuge 14 (such as out of the bottom) and briquetted. The other output may include a liquid output (e.g., clarified liquid), which may continue through a pipe for further processing.

The clarified liquid obtained from the previous step may be subjected to a targeted centrifugation process using centrifuge(s) 16. This targeted centrifugation process is optimized to extract specific valuable components present in the liquid fraction. In other words, a centrifuge may be calibrated to extract a specific compound, and thus, the clarified liquid may be processed in several centrifuges to extract multiple compounds. The components extracted may include, but are not limited to lignin, protein, sugars, omega-3 fatty acids, chlorophyll, and terpenes. Depending on the desired extractions, there may be a preferred sequence of centrifuge processing that the liquid is subject to. In other words, if component 1, component 2 and component 3 are desired to be extracted, it may be preferable to extract component 1 first, then component 2, then component 3 to optimize the extractions and to allow for efficiency. The system may include a database of preferred extraction sequences that may be referenced to identify the preferred extraction sequence. The system may include a controller and one or more processors that may reference the database to determine the optimized sequence in response to identification of a preferred extraction list.

The optimized conditions for each centrifugation step, such as rotation speed, duration, and temperature, are determined to ensure the efficient separation and extraction of the desired components. The resulting extracted components can be collected separately and used for various industrial, nutritional, and commercial applications.

A membrane filtration system may be used to remove water and reduce the liquid to under 50% moisture content leaving behind liquid and a higher percentage of solids. This reduced moisture content substance may be passed through, or otherwise interfaced with a dryer, which may lyophilize (e.g., freeze dry) the biomass to remove water. This process may be repeated depending on the moisture content of the input. Alternatively, this input may be spray dried or dried using an airbed drying mechanism. It is also contemplated that these methods of drying may be used together. The drying method depends on the moisture content of the input and based on the end use of the product/binder.

Various aspects of the present disclosure allow for efficient extraction of valuable components from wet hemp, while minimizing of losses during the processing of hemp and allowing for versatility in extracting multiple valuable components for various applications.

The present disclosure provides a novel and efficient technology for processing wet hemp to extract valuable components such as lignin, protein, sugars, omega-3 fatty acids, chlorophyll, and terpenes. This technology holds promise for applications in industries such as agriculture, nutrition, pharmaceuticals, and more.

The particulars shown herein are by way of example only for purposes of illustrative discussion, and are not presented in the cause of providing what is believed to be most useful and readily understood description of the principles and conceptual aspects of the various embodiments of the present disclosure. In this regard, no attempt is made to show any more detail than is necessary for a fundamental understanding of the different features of the various embodiments, the description taken with the drawings making apparent to those skilled in the art how these may be implemented in practice.

What is claimed is:

1. A method of processing hemp to obtain a desired hemp component, the method comprising the steps of:
providing a liquid solution including hemp into a press;
using the press to separate the liquid solution into a liquid faction and a solid faction, the solid faction including hemp;
using a centrifuge to separate the liquid faction into heavy materials and a clarified liquid; and
separating the clarified liquid into waste and a desired component.

2. The method recited in claim 1, further comprising the step of creating the liquid solution by cutting the hemp to release natural liquids from the hemp.

3. The method recited in claim 2, wherein the liquid solution is created independent of any additives other than the hemp.

4. The method recited in claim 2, wherein the liquid solution includes a liquid component comprised solely of natural liquids in the hemp.

5. The method recited in claim 1, further comprising the step of chopping the hemp into pieces.

6. The method recited in claim 1, wherein the hemp used in the liquid solution has less than 1% cannabidiol (CBD).

7. The method recited claim 1, wherein the press is a screw press.

8. The method recited in claim 1, further comprising the step of directing air under pressure within the press to keep the press clear of obstruction.

9. The method recited in claim 1, wherein the step of providing the liquid solution includes providing chopped hemp into the press by a conveyor.

10. The method recited in claim 1, further comprising the step of setting the press to a specific pressure based on a moisture content of hemp loaded into the press.

11. The method recited in claim 10, further comprising the step of obtaining moisture content data of the hemp loaded into the press from a moisture sensor.

12. The method recited in claim 11, further comprising the step of autonomously setting the press to the specific pressure in response to the moisture content data received from the moisture sensor.

13. The method recited in claim 1, further comprising the step of drying an output of the centrifuge.

14. The method recited in claim 1, wherein the separating step includes using a targeting centrifuge to separate the waste from the desired component.

15. A method of processing hemp to obtain a desired hemp component, the method comprising the steps of:
cutting hemp to release natural fluids from the hemp and produce a wet hemp solution;
pressing the wet hemp solution to generate a liquid fraction and a solid fraction;
using a centrifuge to separate heavy materials from a clarified liquid in the liquid fraction;
using a centrifuge to separate a targeted material from waste; and
passing the targeted material through a membrane filtration system to dry the targeted material.

16. The method recited in claim 15, further comprising the step of subjecting the targeted material to a dryer.

17. The method recited in claim 15, wherein the liquid solution is created independent of any additives other than the hemp.

18. The method recited in claim 15, wherein the hemp used in the liquid solution has less than 1% cannabidiol (CBD).

19. The method recited in claim 15, further comprising the step of directing air under pressure within the press to keep the press clear of obstruction.

20. The method recited in claim 15, wherein the step of providing the liquid solution includes providing chopped hemp into the press by a conveyor.
